(19) United States
(12) Patent Application Publication
(10) Pub. No.: US 2026/0263774 A1
(43) Pub. Date: Sep. 10, 2026

(54) CLOSURE CAP FOR THE STERILIZATION OR FILLING OF A MEDICAL ARTICLE

(71) Applicant: FRESENIUS MEDICAL CARE DEUTSCHLAND GMBH, Bad Homburg (DE)

(72) Inventors: Franz KUGELMANN, Bad Homburg (DE); Andreas RUFFING, Bad Homburg (DE)

(73) Assignee: FRESENIUS MEDICAL CARE DEUTSCHLAND GMBH, Bad Homburg (DE)

(21) Appl. No.: 19/166,492

(22) PCT Filed: Mar. 22, 2024
(86) PCT No.: PCT/EP2024/057779

§ 371 Date: Sep. 18, 2025

(30) Foreign Application Priority Data
Mar. 24, 2023 (DE) 10 2023 202 720.8

(51) Int. Cl. A61M 39/20; A61L 2/07; A61L 2/206; A61L 2/26; A61L 103/15; A61M 1/16; B65B 3/00

(52) U.S. Cl. CPC A61M 39/20; A61L 2/07; A61L 2/206; A61L 2/26; A61M 1/168; B65B 3/003; A61L 2103/15

(57) ABSTRACT

The invention relates to a closure cap for sterilizing or aseptically filling medical articles, in particular for sterilizing dialyzers and treatment sets for dialysis, the closure cap being characterized by a closure plug that is attached to the base body of the closure cap via a releasable or displaceable connection (132) and the closure plug can be moved from an open position for passing through a sterilizing fluid to a tight closed position.

The invention relates to a closure cap for the sterilization or aseptic filling of medical articles, in particular for the sterilization of dialyzers and treatment sets for dialysis. More particularly, the invention relates to a medical article having a fluid port and a closure cap according to the invention.

Medical articles, such as dialyzers and treatment sets, tubing sets for dialysis and especially for extracorporeal blood treatment are used in the treatment of patients in various therapies. A prerequisite for the use of such medical articles in the therapy of patients is their sterility. To ensure the sterility of the medical articles, such medical articles are subjected to a heat sterilization process, in particular a steam sterilization process or a sterilization process with a sterilizing gas, such as such as ethylene oxide. Furthermore, in order to achieve low particle contamination of dialyzers or other medical articles commercially provided, fluid ports of the dialyzers or medical articles need to be closed after having passed the rinsing and sterilization steps in the production process. “Inline” steam sterilization is a widely used sterilization method, particularly for the sterilization of dialyzers in an economic industrial production process. Various embodiments of the “inline” steam sterilization are described in the European patent application EP 0 427 055 A2 and the international patent application WO 2018/104498 A9.

According to the “inline” steam sterilization, the produced dialyzers are mounted to a rinsing and sterilization device via the fluid ports of the dialyzers. The dialyzers are then rinsed in several steps with for example water via the blood ports and dialysate ports of the dialyzer. This process is used to remove unwanted residues originating from the production of dialyzers, e.g. particles, residual solvent or residual polymer, from the inside of the dialyzer. In a further step, the dialyzer is usually sterilized by superheated vapor, at for example 121° C. Accordingly, superheated water vapor is introduced into the interior of the dialyzer via the blood and/or dialysate ports, thus sterilizing the interior of the dialyzer.

For this sterilization procedure, it is necessary to take precautions so that the dialyzers, once removed from the rinsing and sterilization device, cannot be re-contaminated via the blood and dialysate ports remaining opened when the dialyzer is dismounted from the sterilization device. To counteract this, sealing caps were developed to connect a dialyzer to the rinsing and sterilization device via the blood and dialysate ports in a fluid-and germ-tight manner which are functionally so designed to seal the blood and dialysate connections when the dialyzer is removed from the rinsing and sterilization device.

Besides, closure caps for fluid ports of medical articles, particularly dialyzers, are also useful for resealing the medical article after use in a therapeutic procedure. After completion of an extracorporeal blood treatment session, the dialyzer may still be partially filled with treatment fluid and/or blood. For reasons of hygiene, care must be taken to ensure that the treatment site is not contaminated with residual treatment fluids and/or blood from the patient when the dialysis machine and treatment device are dismantled. It is therefore important to reseal the used dialyzer.

In this context, it should be mentioned that commercially available dialyzers usually have two fluid ports for blood. In addition, such dialyzers have at least one fluid port for a treatment fluid or for the filtrate produced in an extracorporeal blood treatment. Generally, the dialyzers addressed in this application have two fluid ports for blood and two fluid ports for a treatment fluid, such as a dialysis fluid. The geometric design of the fluid ports of a dialyzer is specified in standards, such as described in DIN EN ISO 8637:March 2014. However, depending on the therapeutic procedure and the geographical region, not all dialyzers sold are designed according to this standard.

In addition to the requirements of sterilization of medical articles, the process of aseptic filling is also of great importance in medical technology. In the aseptic filling of a medical container, a container to be filled is filled with sterile or aseptically pretreated liquid. Such filling methods can be performed via filling the medical container under aseptic conditions by means of a closure cap.

Patent application EP 2 114 344 A2 describes a closure cap for filling and closing containers containing medical liquids and a method for filling a container with a medical liquid and closing the container, wherein a closure piece is arranged in a closure body of the closure and is displaceable in a releasing position and a position closing the passage of the closure body.

Patent application EP 0 352 540 A2 describes a multi-part closure cap for fluid ports of a dialyzer, which consists of a first and a second cap part, wherein the first cap part is attachable to the fluid port of the dialyzer and the second cap part is movable between an open position, in which the passage of a sterilization medium takes place, and a closing movement.

### OBJECT OF THE INVENTION

Based on the closure caps described in the prior art and considering the requirements of existing processes for sterilizing and aseptic filling of medical articles, there is a need to develop alternative closure caps that are optimized in terms of manufacturing, handling and functionality and are functionally so designed that they can be used in a sterilization process or aseptic filling process and can seal the sterilized or filled medical article in a fluid-tight and thus germ-tight manner.

### SUMMARY OF THE INVENTION

In a first aspect of the invention, the problem is solved by a closure cap having the features of claim 1. Dependent claims 2 to 13 provide preferred embodiments.

In a second aspect of the invention, the problem is solved by a medical article comprising a fluid port and a closure cap according to any of the claims of 1 to 13.

In a third aspect of the invention, the problem is solved by using a closure cap having the features according to any one of claims 1 to 13 for sterilization or aseptic filling.

### DESCRIPTION OF THE INVENTION

The closure cap described according to the first aspect of the invention is suited to be placed on a fluid port of a medical article. In an open position of the closure cap, the closure cap is suitable for introducing a fluid into the medical article. The fluid may be a sterilizing medium, such as a gas such as ethylene oxide or superheated water vapor at 121° C., or may be an aseptically or sterilized filling material, such as a medical liquid. In a closed position, the closure cap is suitable for closing the fluid port of the medical article in a fluid-and germ-tight manner, in particular, to close the fluid ports of a dialyzer and to ensure sterility and low particle contamination of the dialyzer. In particular, the closure cap is configured to cooperate with a first fluid port of a dialyzer in a fluid-tight and/or liquid-tight manner. Provided that the connection between the closure cap and the fluid port of the dialyzer is fluid-or liquid-tight, this connection is also germ-tight and sterility and low particle contamination are guaranteed. In particular, the closure cap may also be configured to cooperate in a fluid-tight manner with the blood port and/or a dialysate port of a dialyzer for extracorporeal blood treatment.

Furthermore, the closure cap according to the invention is also suited to cooperate as a connector between a first fluid port of a medical article, in particular a dialyzer, and a rinsing and sterilization device, in particular a device for “inline” sterilization. The closure cap according to the first aspect simplifies the process of an industrial rinsing and sterilization procedure. The closure cap has a receiving portion via which a fluid-sealing connection can be made to a first fluid port of a medical article, in particular a dialyzer. Furthermore, the closure cap according to the invention can be connected to a rinsing and sterilization device to introduce a sterilization medium into the medical article in an open position of the closure cap, in particular to allow “inline” sterilization processes or a sterilization process with a sterilizing gas. Alternatively, the closure cap can also be used to connect to a filling station in an open position of the closure cap in order to fill a medical article, such as a medical container with a medical liquid.

Open position and closed position of the closure cap according to the invention are enabled by a closure plug releasably attached to the closure cap. In the open position, the closure plug comprises a fluid channel through the sealing cap through which a sterilization medium or a medical filling material can be flowed. In the closed position of the closure plug, the fluid channel is blocked in a fluid-tight manner by the closure plug. The closure plug can be moved from the open position to the closed position by applying a force. Appropriate means can be provided on the rinsing and sterilization device to move the closure plug from an open position to a closed position.

According to the present invention, the handling of the closure caps is also simplified during therapeutic procedures. When using the closure caps according to the invention on a dialyzer in an extracorporeal blood treatment process, they are removed immediately before an extracorporeal blood treatment of the dialyzer. Usually, after a patient has been treated with blood, it is then necessary to close the dialysate ports of the dialyzer to prevent spilling of residual dialysate out of the dialyzer. The closure caps according to the invention can be used for this purpose. In one embodiment, the closure cap may be configured to provide a fluid-tight seal to both the dialysate port and the blood port of a dialyzer. This enables improved handling of the closure caps according to the invention while setting up and dismantling a dialysis machine in course of conducting an extracorporeal blood treatment.

Further details, features and advantages of the invention will be apparent from the following description and drawings. It is shown in:

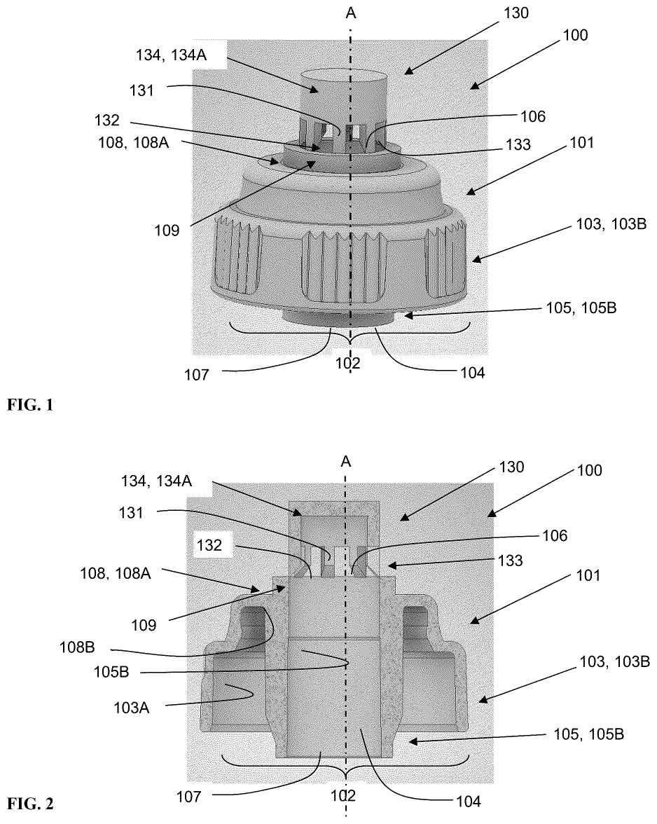

FIG. 1 a schematic representation of a side view of a closure cap according to the invention in the open position, in which the closure plug is releasably attached to the base body of the closure cap,

FIG. 2 a sectional drawing of a closure cap according to the invention along a central longitudinal axis A in the open position of the closure cap, in which the closure plug is releasably attached to the base body of the closure cap,

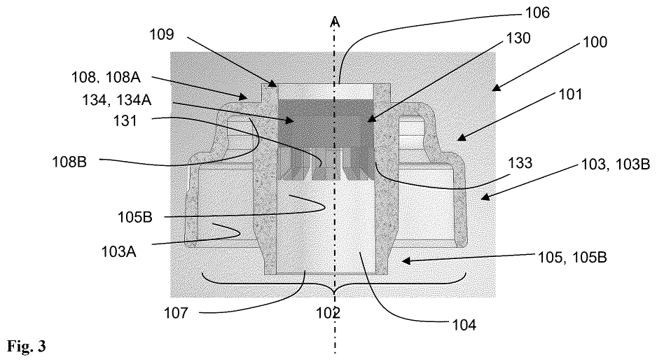

FIG. 3 a sectional drawing of a closure cap according to the invention along a central longitudinal axis A in the closed position of the closure cap in which the closure plug blocks the fluid channel of the closure cap in a fluid-tight manner.

### DETAILED DESCRIPTION OF THE INVENTION

In the following, the invention is described with reference to individual embodiments which are illustrated in FIG. 1 to 3. Individual embodiments herein described are not limited to the embodiments shown in FIGS. 1 to 3.

In a first aspect, the invention relates to a closure cap 100 for a fluid port of a medical article, in particular for a fluid port of a dialyzer, comprising a base body 101 and a closure plug 130, wherein the base body and the closure plug are arranged along a central longitudinal extension axis A and connect to each other, the base body 101 comprising a circumferential side wall 103 having an inner side 103A and an outer side 103B, an end wall 108 having an inner side 108A and an outer side 108B, a fluid channel 104 arranged centrally symmetrically with respect to the first circumferential side wall 103, comprising a circumferential wall 105 having an inner side 105A directed towards the fluid channel, and an outer side 105A directed towards the inner side 103A of the fluid channel, and an outer side 105B directed towards the inner side 103A of the circumferential side wall 103, a first open end 106 and a second open end 107 for passing fluids, the circumferential side wall 103 being circumferentially connected to the outer side 105B of the wall 105 of the fluid channel 104 via the end wall 108 in a fluid-tight manner, characterized in that the closure plug 130 is detachably or slidably attached to the base body, and the closure plug 130 is dimensioned such that the closure plug 130 can be inserted into the fluid channel 105 from an open position in which a fluid can flow into the fluid channel through the first open end 106 to a fluid-sealing closed position.

In the context of the present invention, a “dialyzer” is understood to mean a hollow fiber membrane filter for extracorporeal blood treatment having at least two fluid ports for blood and at least one other fluid port for dialysate or filtrate. In the context of the present invention, the term dialyzer refers to such hollow fiber membrane filters used in extracorporeal blood treatment. In particular, in the context of the present application, the term “dialyzer” also includes hollow fiber membrane filters used as blood treatment filters in dialysis, hemodiafiltration, hemofiltration, plasma separation, or blood oxygenation.

For the purposes of the present application, the term “closure cap” is understood to mean a closure element that closes an opening of a fluid port of a medical article in a fluid-tight manner in a closed position. For the purposes of the present application, “fluid-tight” is understood to mean that a fluid, e.g. a rinsing liquid or a sterilization gas, such as e.g. superheated water vapor, cannot pass through a component section of the closure cap, or can escape through an opening closed by the closure cap under use.

For the purposes of the present application, the term “superheated steam” is used equivalently to the technically common terms “moist heat” or “saturated steam”. For the purposes of the present application, this refers to gaseous water with a temperature above 100° C. Additionally, during steam sterilization of medical articles, pressure is applied to accelerate and improve the sterilization process. Such pressures range from 1 to 5 bar. For the purposes of the present application, the terms “superheated water vapor”, “moist heat” or “saturated steam” are also understood to mean gaseous water at a temperature of 110 to 130° C., in particular 121° C., and a pressure of 1 to 5 bar, in particular also 2.5 bar.

Within the context of the present application, the term “base body” is understood to mean a component portion of the closure cap which encloses a receiving portion for a fluid port of a medical article, i.e. which is in particular suited to cooperate in a fluid-tight manner with a fluid port of a medical article.

For the purposes of the present application, the term “circumferential sidewall” means a sidewall that extends along a circumference. That means that a line can be mapped circumferentially on the sidewall, which is not interrupted. Sections of the circumferential sidewall may have an inner or outer surface that is substantially cylindrical in shape. The circumferential sidewall may further include shoulders, protrusions, and cross-sectional protrusions. In certain embodiments, the circumferential side wall is geometrically configured to cooperate in a fluid-tight manner with the fluid port of a medical article, particularly a dialysate port of a dialyzer.

For the purposes of the present application, the term “end wall” is understood to mean a component section which connects the circumferential side wall circumferentially and, in a fluid-tight manner to the centrally symmetrically arranged fluid channel.

For the purposes of the present application, the term “receiving portion” is understood to mean a section of the closure cap into which a fluid port of a medical article, in particular the dialysate port or the blood port of a dialyzer, can be inserted in a fluid-tight manner. The receiving portion can be formed by the section of the circumferential side wall, the wall of the fluid channel and the end wall, wherein the wall of the fluid channel can then be inserted into the opening of a fluid port of a medical article, in particular the opening of the dialysate port of a dialyzer. Alternatively, however, the closure cap can be configured such that the fluid channel can be connected to a geometrically different fluid port of a medical article, e.g. can accommodate the blood port of a dialyzer.

The term “closure plug” is understood to mean a component body that can be fully or partially inserted into the fluid channel of the closure cap and can thus block the fluid channel in a fluid-tight manner. In the base configuration of the closure cap, i.e. after manufacture, the closure plug initially connects releasably to the base body of the closure cap. In this state, the one fluid channel arranged centrally symmetrically with respect to the first circumferential side wall can be passed by a fluid flowing therethrough via the first open end and the second open end of the fluid channel. Provided that a fluid can flow through the fluid channel, the closure plug and thus also the sealing cap are in an “open position” within the meaning of the present application. Provided that the closure plug has been inserted in a fluid-sealing position in the fluid channel, the closure plug and thus also the sealing cap is in a “closed position” within the meaning of the present application.

Within the context of the present application, the term “releasable connection” is understood to mean a connection consisting of the base body and the closure plug which can be released with a defined force. In particular, a releasable connection can also be understood as a predetermined breaking point, according to which a breakage of the connection is possible with a defined force without the closure cap being impaired in its function. It can also be provided that the connection is slidable. Such embodiments have a material of the connection of the closure plug and the base body with particularly high ductility.

A central longitudinal extension axis is understood to be a geometric axis of the closure cap which runs centrally through the centrally symmetrically arranged fluid channel and defines a longitudinal extension of the closure cap in terms of the translatory movement of the closure plug into the centrally symmetrically arranged fluid channel from an open position to a closed position.

In another embodiment of the first aspect of the invention, the closure cap is characterized in that the closure plug is removably attached to the end wall 108, in particular the outer surface 108A of the end wall 108 or to the first open end of the fluid channel 106 on the circumferential wall 105. A corresponding embodiment is shown in FIGS. 1 and 2.

In a further embodiment of the first aspect of the invention, the closure plug is characterized in that the closure plug has a section with lateral passage openings 131 through which a fluid can flow in the open position of the closure plug and which, in a closed position, are covered in a fluid-tight manner by the inside of the wall of the fluid channel. According to this embodiment, fluid guidance in the open position of the closure cap is enabled through the fluid channel by the lateral openings in the closure plug. Advantageously, the closure plug can be inserted into the fluid channel 104 and the lateral openings are covered by the inner side 105A of the wall 105 of the channel 104 in a fluid-tight manner in the closed position. In particular, in this embodiment, the closure plug may be recessed into the channel 104.

In another embodiment of the first aspect of the invention, the closure cap is characterized in that the wall 105 of the fluid channel 104, which is centrally symmetrical with respect to the first circumferential side wall 103, has a substantially cylinder-shell-shaped inner surface 105A. According to this embodiment, in the sense of a fluid-and germ-tight closed position of the closure plug in the fluid channel, the closure plug is also designed in sections to be cylindrical in such a way that the closure plug can be inserted into the channel in a fitting and fluid-tight manner. In particular, the cylindrical shape of the fluid channel and the closure plug is advantageous in terms of a germ-tight sealing between the closure plug and the inner side 105A of the wall 105 of the fluid channel 104.

In particular, according to this embodiment, the closure plug is configured such that the closure plug comprises a cylindrical portion 134 having an outer surface 134A which, in the closed position of the closure plug, cooperates in a fluid-tight manner with the cylinder-shell-shaped inner surface 105A of the wall 105 of the fluid channel 104 arranged centrally symmetrically with respect to the first circumferential side wall 103.

In another embodiment of the first aspect of the invention, the closure cap 100 is characterized in that, in the open position of the closure plug, the closure plug 130 connects to the wall 105 at the first open end of the fluid channel 106 via one or more portions of a releasable connection 132. According to one embodiment, the releasable connection may be formed by circumferentially connecting to the wall at the first open end of the fluid channel 106. In alternative embodiments, the circumferential releasable connection may be interrupted into multiple sections of releasable connections. Where multiple sections of releasable connections are circumferentially connected to the wall 105 at the first open end of the fluid channel 106, a lateral opening may advantageously be provided on the closure plug between each section.

In another embodiment of the first aspect of the invention, the closure cap 100 is characterized in that, when the closure plug is in the open position, the closure plug 130 has a plurality of circumferential pinnacles (133) laterally connecting circumferentially via sections of releasable connections 132 to the wall 105 at the first open end of the fluid channel 106.

In another embodiment of the first aspect of the invention, the closure cap 100 is characterized in that the one or more sections of a releasable connection 132 are releasable with a force of from 10 to 100 N, more particularly from 20 to 60 N, furthermore particularly from 25 to 45 N. In particular, according to this embodiment, the releasable connections are sufficiently stable to allow the closure caps to withstand the mechanical and thermal requirements in rinsing and sterilization processes or, alternatively, in filling processes. In addition, the force is such that the closure caps can be handled in their functionality in terms of the above-mentioned processes.

In another embodiment of the first aspect of the invention, the closure cap 100 is characterized in that the one or more portions of a releasable connection 132 comprises an adhesion connection, a snap connection, or a weakening of material. In terms of an adhesive connection, sticking or heat-sealed connections are particularly suitable. Particularly advantageously, the releasable connection consists of a material weakening that can be broken with a defined force. This has the advantage that, in the open position, the closure cap can be worked as a one-piece closure cap, for example the closure cap can then be manufactured in an injection molding process, which represents an economic preferred manufacturing process. Furthermore, the closure cap can be in two parts in the closed position.

In another embodiment of the first aspect of the invention, the closure cap 100 is characterized in that the inner surface 105A of the wall 105 of the fluid channel 104 includes a shoulder that reduces the clear width of the fluid channel 104. The shoulder thus forms a stop, defining a depth of penetration of the closure plug 130 into the fluid channel 104 in the closed position. Due to the defined penetration depth of the closure plug, it can be already visually recognized that the closure plug has reached a sufficient penetration depth in the fluid channel 104 and thus that the sealing cap fulfills the requirements of fluid and germ tightness in the closed position.

In another embodiment of the first aspect of the invention, the closure cap 100 is characterized in that the closure cap includes an annular shoulder 109 on the outer surface 108B of the end wall. Preferably, the one or more sections of the releasable connections 132 connect to this annular shoulder. This embodiment is shown in FIGS. 2 and 3. The annular shoulder advantageously serves to establish a fluid connection to a fluid port of a filling, rinsing or sterilization device. In this embodiment, the annular shoulder 109 may be received in the opening of a fluid port of a filling, rinsing, or sterilizing device, thereby providing a fluid and germ-tight connection between the closure cap and the filling, rinsing, or sterilizing device. Accordingly, a fluid can be introduced into the fluid channel 104 and further into the medical article, in particular into the dialyzer, via the filling, rinsing or sterilization device when the closure cap is in the open position. After completion of the filling, rinsing or sterilization steps, the closure plug can be brought into the closed position by means of a punch tool, which can be part of the filling, rinsing or sterilization device. In this process, the one or more releasable connections between the base body and the closure plug are broken and the closure plug is inserted into the channel 104.

In another embodiment of the first aspect of the invention, the closure cap 100 is characterized in that the receiving portion 102 is geometrically configured to cooperate in a fluid-tight manner with a blood port and/or a dialysate port according to DIN EN ISO 8637:March 2014 of a dialyzer.

In another embodiment of the first aspect of the invention, the closure cap 100 is characterized in that the closure cap is made of a plastic material selected from the group of thermoplastics, in particular polyethylene, polypropylene or thermoplastic elastomers or blends or copolymers thereof. Polypropylene homopolymers and copolymers are particularly preferred. Such materials are dimensionally stable, particularly in the application of heat sterilization, and therefore suitable for germ-and fluid-tight cooperation with a fluid port of a medical article, particularly for the cooperation with the dialysate and blood ports of a dialyzer, under high temperatures.

In a second aspect, the invention relates to a medical article comprising at least one fluid port provided with a closure cap according to one of the embodiments according to the first aspect. The medical article may be, in particular, a dialyzer a tubing set for dialysis, a treatment set for dialysis, or a container for holding medical fluids.

Furthermore, in a third aspect, the invention relates to the use of a closure cap according to any of the embodiments according to the first aspect for rinsing, sterilizing or aseptically filling a medical article.

### REFERENCE SIGNS OF THE FIGURES

A Longitudinal extension axis
100 closure cap
101 base body of the closure cap
102 receiving portion to cooperate with a fluid port
103 circumferential side wall
103A inner side of the circumferential side wall
103B outer side of the circumferential side wall
104 fluid channel
105 circumferential wall of the fluid channel
105A inner side of the circumferential fluid channel
105B outer side of the circumferential fluid channel
106 first open end of the fluid channel
107 second open end of the fluid channel
108 end wall of the circumferential side wall
108A inner side of the end wall of the circumferential side wall
108B outer side of the end wall of the circumferential side wall
109 annular shoulder
130 closure plug
131 lateral openings at the closure plug
132 releasable connection between base body and closure cap
133 pinnacles of the closure cap
134 cylindrical section of the closure plug
134A outer side of the cylindrical section of the closure plug

1. A closure cap for a fluid port of a medical article comprising a base body and a closure plug, wherein the base body and the closure plug are arranged along a central longitudinal extension axis and are connected to each other,
the base body comprising,
a circumferential side wall having an inner side and an outer side, an end wall, having an inner side and an outer side, a fluid channel arranged centrally symmetrically with respect to the first circumferential side wall, comprising a circumferential wall having an inner side facing the fluid channel and an outer side facing the inner side of the circumferential side wall, a first open end and a second open end for passing fluids,
wherein the circumferential side wall is circumferentially, fluidly sealingly connected to the outside of the wall of the fluid channel via the end wall, wherein
the closure plug is attached to the base body via a releasable or slidable connection, and the closure plug is sized such that the closure plug is insertable into the fluid channel from an open position in which a fluid can flow into the fluid channel through the first open end to a fluid-sealing closed position.

2. The closure cap of claim 1, wherein the closure plug is removably attached to the circumferential wall at the end wall or at the first open end of the fluid channel.

3. The closure cap according to claim 1, wherein that the closure plug has a section with lateral passage openings through which a fluid can flow in the open position of the closure plug and which, in a closed position, are covered in a fluid-tight manner by the inside of the wall of the fluid channel.

4. The closure cap according to claim 1, wherein the wall of the fluid channel arranged centrally symmetrically with respect to the first circumferential side wall has a substantially cylinder-shell-shaped inner side.

5. The closure cap according to claim 4, wherein the closure plug comprises a cylindrical portion having an outer surface which, in the closed position of the closure plug, cooperates in a fluid-tight manner with the inner cylindrical shell surface, of the wall of the fluid channel arranged centrally symmetrically with respect to the first circumferential side wall.

6. The closure cap according to claim 1, wherein in the open position of the closure plug, the closure plug connects to the wall at the first open end of the fluid channel via one or more portions of a releasable connection.

7. The closure cap of claim 6, wherein in the open position of the closure plug, the closure plug has a plurality of circumferential pinnacles laterally connecting circumferentially via sections of releasable connections to the wall at the first open end of the fluid channel.

8. The closure cap of claim 6, wherein the one or more portions of a releasable connection are releasable with a force of 10 to 100 N.

9. The closure cap according to claim 6, wherein the one or more portions of a releasable connection consists of an adhesive connection, a snap connection, or a weakening of material.

10. The closure cap according to claim 1, wherein the inner surface of the wall of the fluid channel has a shoulder that reduces the clear width of the fluid channel.

11. The closure cap according to claim 1, wherein the closure cap is a one piece closure cap in the open position.

12. The closure cap according to claim 1, wherein the wall of the fluid channel projects beyond the end wall and forms an annular shoulder on the end wall of the closure cap.

13. The closure cap according to claim 1, wherein the receiving portion is geometrically configured to cooperate in a fluid-tight manner with a blood port and/or a dialysate port according to DIN EN ISO 8637:March 2014 of a dialyzer.

14. A medical article comprising at least one fluid port provided with the closure cap according to claim 1.

15. A method for for rinsing, sterilizing or aseptically filling a medical article, said method comprising the step of utilizing the closure cap of claim 1.

16. The closure cap according to claim 1, wherein said medical article is a fluid port of a dialyzer.
